(19) United States
(12) Patent Application Publication
(10) Pub. No.: US 2020/0260950 A1
(43) Pub. Date: Aug. 20, 2020

(54) AMD Diagnostic Multi Tester

(71) Applicant: Daniel Joseph Arnow, New Rochelle (WE)

(72) Inventor: Daniel Joseph Arnow, New Rochelle (WE)

(21) Appl. No.: 16/793,863

(22) Filed: Feb. 18, 2020

Related U.S. Application Data
(60) Provisional application No. 62/852,979, filed on May 25, 2019.
(60) Provisional application No. 62/805,987, filed on Feb. 15, 2019.

(51) Int. Cl. A61B 3/12; A61B 3/00

(52) U.S. Cl. CPC A61B 3/12; A61B 3/0075; A61B 3/0091

(57) ABSTRACT

An eye testing system for diagnosing age-related macular degeneration of a patient is disclosed. The eye testing system includes a plurality of acuity charts arranged in a plurality of rows. The each acuity chart includes a plurality of rows of symbols. Further, the eye testing system includes a bulls eye configured to slide relative to the plurality of acuity charts. The bulls eye includes a telescopic arm and an eye portion attached to the telescopic arm to facilitate a viewing of the one or more symbols to facilitate in diagnosing a central vision and a peripheral vision of the eye of the patient.

### TECHNICAL FIELD

The present disclosure relates, generally to an eye testing system for determining age related macular degeneration (AMD) disease in a patient. More particularly, the present disclosure pertains to the eye testing system for diagnosing three symptoms associated with the AMD disease: central vision deterioration and poor dark adaption as measured by perception of a point of light or by perception of figures on a chart The multiplicity of test in one system provides redundancy.

### BACKGROUND

Age-related macular degeneration (AMD) disease affects approximately 20 million people worldwide by affecting a central vision and a peripheral vision of a patient. The disease is caused due to the leakage of blood from the blood vessels directly affecting the vision of the patient. In long term the blood being caustic to a retina vision receptor results into loss of central vision and may lead to complete blindness if not treated. However, a treatment for the AMD disease arrests the bleeding but the caustic damage caused due to the bleeding from the blood vessels cannot be reversed, so the damage done to the retina cells is irreversible. Therefore, the early diagnostics of the AMD disease is essential for starting the treatment at an early stage and reducing the caustic effects.

### SUMMARY

The application herein utilizes the basic machinery originally presented in U.S. Pat. No. 10,251,542 (Arnow'542) issued and published on Apr. 9, 2019 consisting of an eyepiece with light filter plates arranged in a filter rack and an objective basically similar an Optometrists Acuity Chart used to perform Dark Adaption testing. Certain embellishments of that basic structure are presented herein in a new embodiment wherein a Field of Vision test may be accomplished to determine loss of central vision. The text of Arnow'542 is incorporated by reference here.

According to an aspect of the disclosure an eye testing system for diagnosing age-related macular degeneration of a patient is disclosed. The eye testing system includes a plurality of four acuity charts arranged in a plurality of rows. Each acuity chart includes a plurality of rows of symbols. Further, the eye testing system includes a bullseye configured to slide relative to the plurality of acuity charts. The bulls eye includes a telescopic arm and an eye portion attached to the telescopic arm to facilitate a viewing of the one or more symbols to facilitate in diagnosing a loss of central vision of the patient.

In an embodiment, the four-part chart may be replaced by an ordinary Snellen Chart in order to perform a Dark Adaption Test by measuring loss of acuity in darkened conditions as mentioned above in the Technical Field Section.

In an embodiment, the telescopic arm includes a plurality of shafts adapted to telescopically move relative to each other to move the eye portion across a width of the acuity charts.

In an embodiment, the bullseye is adapted to move in a direction perpendicular to a direction of telescopic movement of the telescopic arm.

In an embodiment, the eye testing system further includes a controller to control a telescopic movement of the telescopic arm and a sliding movement of the bullseye relative to the acuity charts.

In an embodiment, the eye testing system further includes a light source disposed above the plurality of acuity charts. The light source exposes an eye of a patient to light to facilitate in performing a dark adaptation test of the eye of the patient.

In an embodiment, the light source is a constant intensity light source.

In an embodiment, a level of intensity of light reaching to the eye of the patient is sequentially reduced by using a plurality of filters to perform the dark adaptation test of the eye.

In an embodiment, a level of intensity of light reaching to the eye of the patient is sequentially reduced by using a rheostat to perform the dark adaptation test of the eye.

In an embodiment, the eye testing system includes a controller to control the rheostat to sequentially vary the level of intensity of light reaching the eye.

In an embodiment, the light source is a variable intensity light source.

In an embodiment, a level of intensity of light reaching to the eye of the patient is sequentially reduced by varying an intensity of light emitted by the light source to perform the dark adaptation test of the eye.

In an embodiment, the eye testing system includes a controller to control the light source to vary the intensity of light emitted by the light source

In an embodiment, the plurality of acuity charts and the Snellen Chart are digital charts such as may be presented on a Computer and Monitor.

In an embodiment, the hand-held filter eyepiece as described above and in Arnow'542 may be strapped to the head with a forehead interface or attached to the front face of an industrial, eye-protective google.

For a more complete understanding of the present invention, reference is made to the following detailed description and accompanying drawings. In the drawing, like reference characters refer to like parts throughout the views in which.

### BRIEF DESCRIPTION OF THE DRAWINGS

FIG. 1 illustrates an eye testing system for diagnosing age related macular degeneration diseases in a patient, in accordance with an embodiment of the disclosure.

### DETAILED DESCRIPTION

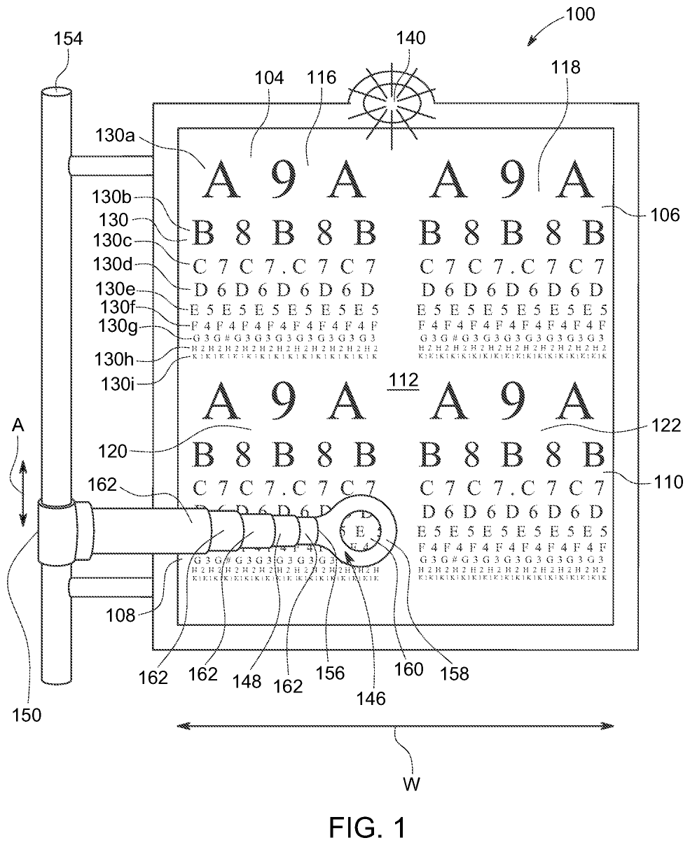

Referring to FIG. 1, an eye testing system 100 suitable to facilitate diagnosis of one or more age related macular degeneration diseases is shown. The eye testing system 100 includes a plurality of acuity charts, for example, a first acuity chart 104, a second acuity chart 106, a third acuity chart 108, and a fourth acuity chart 110, displayed or arranged on a single surface 112. As shown, the surface 112 may be a rectangular surface, the four acuity charts 104, 106, 108, 110 are displayed or mounted on the surface 112 such that each quadrant of the surface 112 includes a single acuity chart. For example, the first acuity chart 104 is displayed on a first quadrant 116 of the surface 112, the second acuity chart 106 is displayed on a second quadrant 118 of the surface 112, the third acuity chart 120 is displayed on a third quadrant 120 of the surface 112, and the fourth acuity chart 122 is displayed on the fourth quadrant 122 of the surface 112. In this manner, the four acuity charts 104, 106, 108, 110 are arranged in two rows, wherein each row includes two acuity charts arranged spaced apart from each other. In an embodiment, the surface 112 may be a surface of a plate adapted to hang on a wall. In certain implementations, the surface 112 may be a front surface of an optical box illuminated by one or more lights. Alternatively, the surface 112 may be a display screen of a computer displaying the four acuity charts 104, 106, 108, 110 on the display screen of the computer. In certain embodiments, the four acuity charts 104, 106, 108, 110 may be projected on a surface using a projector. Further, it may be appreciated that a structure of the second acuity chart 106, a structure of the third acuity chart 108, and a structure of the fourth acuity chart 110 are similar to a structure of the first acuity chart 104, and for the sake of clarity and brevity, only the structure of the first acuity chart 104 is explained.

The first acuity chart 104 includes a plurality of rows 130 including a plurality of symbols arranged linearly in each of the plurality of rows 130. In an embodiment, the symbols may include a plurality of letters and/or a plurality of numerals. In an embodiment, the symbols are arranged in a single row 130 are of equal size, while the sizes of the symbols in different rows are different. For example, the sizes of the symbols arranged in a first row 130a, defining the topmost row, are larger than the symbols arranged in any other row. Further, the sizes of the symbols arranged in a row 130, for example, a second row 130b, is smaller than the sizes of the symbols arranged in the row disposed above and adjacent to the row, for example, the first row 130a. The size of the symbols on the chart are adjusted so that they correspond to the acuity abilities of a patient with 20/20 vision, a patient with 20/30 vision, et cetera, at the test distance of only 4 feet so to provide an appropriate range of vision for the Field of Vision test. As an example, the first acuity chart includes 9 rows including the first row 130a having the one or more than one symbol shaving a visual acuity equivalent of 20/200, the second row having the one or more than one symbols having a visual acuity equivalent of 20/100, a third row 130c having the one or more than one symbols having a visual acuity equivalent of 20/80, a fourth row 130d having the one or more than one symbols having a visual acuity equivalent of 20/60, a fifth row 130e having the one or more than one symbols having a visual acuity equivalent of 20/50, a sixth row 130f having the one or more than one symbols having a visual acuity equivalent of 20/40, a seventh row 130g having the one or more than one symbols having a visual acuity equivalent of 20/30, an eighth row 130h having the one or more than one symbols having a visual acuity equivalent of 20/25, and a ninth row 130i having the one or more than one symbols having a visual acuity of 20/20. In this manner, the sizes of the symbols of each row decreases from the first row 130a (topmost row) to the last row 130i (bottommost row) of the first acuity chart 104.

Further, the eye testing system 100 may include a light source 140 to expose an eye of a patient with light for facilitating the testing of the eye against the light source 140. In an embodiment, the light source 140 may emit a constant intensity of light. The light source 140 emits a quantifiable unit of luminance for measuring the light sensitivity of the eye. It may be appreciated that the light source 140 may include a light bulb and/or circuitry which produces a light brightness which is consistent and not affected by current variation and/or bulb aging. Alternatively, the light source 140 may be controlled to provide light of different intensities. In an embodiment, the surface 112 is a vertical plate, and in such a case, the light source 140 may be arranged/mounted proximate to a top end or top edge of the vertical plate. In an embodiment, the light source 140 may be a lamp or a light emitting diode.

Moreover, the eye testing system 100 may include a bullseye 146 adapted to slide in a direction ‘A’ relative to the surface 112 and facilitates in performing a central vision and peripheral vision test of a patient. The bulls eye 146 may include a telescopic arm 148 having a first end 150 that may be coupled to a vertical post 154 and a second end 156 having an eye portion 158 defining a hole 160 to facilitate a viewing of the one or more symbols on one of the four acuity charts 104, 106, 108, 110. As shown, the bullseye 146 is adapted to slide along a length of the vertical post 154 to position one or more desired symbols into the eye portion 158. As shown, the telescopic arm 148 may include a plurality of shafts 162 adapted to extend or retract relative to each other to facilitate a movement of the eye portion 158 along a width ‘W’ of the surface 112, i.e. in a direction perpendicular to the direction ‘A’. In this manner, the bullseye 146 facilitates in focusing on any of the symbols of the four acuity charts 104, 106, 108, 110 In an embodiment, the hole 160 includes a size that corresponds to 10 percent to 13 percent of a total area of the field of vision.

In an embodiment, the eye testing system 100 and the eyepiece of the system are interspaced with magnifying lenses so to shrink the focal distance between surface 112 having the four acuity charts 104, 106, 108, 110 and the eyepiece of the system so that the entire system can fit under one machinery cover. In an embodiment, the eye testing system 100 may include a controller to control the level of intensity of the light source 140, a movement of the bulls eye 146 along the direction ‘A’, a telescopic movement of the telescopic arm 148 by controlling the extension and/or retraction of the plurality of shafts 162, and the eye piece lens and the objective lens to control the distance between the four acuity charts 104, 106, 108, 110 and the patient's eye. In an embodiment, the controller may vary an intensity of the light source 140 to dim the light emitted by the light source 140 to check dark adaptation of the eyes of the patient. In certain implementations, the controller may control a rheostat reducing the electric current automatically and sequentially to the light source 140 to vary the intensity of light reaching the eye of the patient and therefore perform the dark adaptation test for the patient. In certain scenarios, the controller may be used to electively choose the light intensity. When a rheostat is not utilized to control the light intensity, a hand-held eyeglass clip or dark adaptation tester disclosed in Arnow'542 may be utilized to sequentially vary the intensity of light reaching the eye of the patient to perform the adaptation test. In certain embodiments, the four acuity charts 104, 106, 108, 110 along with the light source 140 and the bullseye 146 may be mounted on a vertical wall to conduct various eye vision tests. The bullseye may also be hand held by the test administrator.

The objective assembly herein becomes operational and useful when used in conjunction with the filter-rack eyepiece consisting of an occlude base plate with a multi-place filter rack designed to hold neutral density filters of varying densities, the rack being reversable so to alternate left and right eye for testing, as is more fully described in Arnow'542. This eyepiece may also be affixed to the head with two or more sterilizable straps and a sterilizable forehead interface between the forehead and the occlude base plate or by attaching the filter eyepiece to the front of a headpiece similar to an industrial eye protection google. Optical instruments have often been presented in the prior art with the alternate format of hand-held (lorgnette) or head affixed units.

Methods of testing or diagnosing the eye of the patient for various age-related macular degeneration (AMD) using the eye testing system 100 in now explained. The patient suffering from the AMD disease will show weakened ability to see when the light is going from bright to dim and will have areas of poor central vision. For testing the eye of the patient for dark adaption, the patient is maintained at a distance of about 20 feet from the Snellen Acuity Chart and the light source 140 emitting constant intensity of light. The patient is required to recognize the symbols on the Acuity Chart or the point of light in darkened conditions to establish the threshold of blindness The intensity of light is sequentially or progressively reduced, by reducing the intensity of the light using rheostat or by using filters of progressive intensity of darkness, or by controlling the light source 140. The threshold level of blindness is determined in which the patient is not able to recognize the symbol in the acuity chart or not able to see the point of light source If the threshold of blindness is quickly reached as the light grows dimmer in one eye than the other, that eye may be affected by AMD disease.

Additionally, the eye testing system 100 may be utilized for testing a central vision and a peripheral vision of the patient required for diagnosing the patient for AMD disease. For testing the eye, the acuity charts is maintained at a certain distance, for example 4 feet, from the eyes of the patient. The bullseye 146 is moved to position one or more symbols inside the eye portion 158 and thereby to focus one or more letters, to be recognized by the patient, from the first row 130a to the ninth row 130i in a sequential manner or otherwise. The central vision of the patient corresponds to the symbols of a smallest letter row that the patient is able to recognize. For determining the peripheral vision of the patient, the eye portion 158 may be positioned on any one or more symbols in the ninth row 130i, then the patient is asked to look at all the symbols arranged around outside the eye portion 158 utilizing his side or peripheral vision as much as possible and recognize the smallest number symbol arranged around outside the eye portion 158 that he can see. If the patient is unable to recognize small symbols in the central eye and able to recognize small symbols on the periphery, the patient has damaged central vision indicative of AMD disease. In the method herein, letters are used for the central vision and numbers are sued for the pheripheral vision to avoid unconscious faulty sightings. Also, the patient may be asked for any blurred area in which the patient may not be able to recognize the symbols in any four acuity charts 104, 106, 108, 110. If there are any blurred areas, the improper peripheral vision corresponding to the blurred area is suggested much as in an Amsler Grid test. The results may be quantified with the special chart herein by comparing line of acuity vision centrally to that peripherally. For example, if the vision is 20/60 equivalent centrally and 20/30 equivalent at the edges we may formulate an index for central vision of 60/20 or 2. An index of appreciably above 1 is suggestive of central vision loss and possible AMD disease, especially the patient is in the age categories where AMD is most prevalent.

The foregoing descriptions of specific embodiments of the present disclosure have been presented for purposes of illustration and description. They are not intended to be exhaustive or to limit the present disclosure to the precise forms disclosed, and obviously many modifications and variations are possible in light of the above teaching. The exemplary embodiment was chosen and described in order to best explain the principles of the present disclosure and its practical application, to thereby enable others skilled in the art to best utilize the present disclosure and various embodiments with various modifications as are suited to the particular use contemplated.

This application claims benefit of U.S. Provisional Patents 62/805,987 and 62/852,979.

What is claimed is:

1. An eye testing system for diagnosing age-related macular degeneration of a patient, the eye testing system comprising:
a plurality of acuity charts arranged in a plurality of rows, each acuity chart includes a plurality of rows of symbols; and
a bullseye configured to slide relative to the plurality of acuity charts and having a telescopic arm and an eye portion attached to the telescopic arm to facilitate a viewing of the one or more symbols to facilitate in diagnosing a central vision and a peripheral vision of the eye of the patient; and
the separated filter rack eyepiece as described herein and in Arnow'542 so to make a complete eyepiece and objective system for AMD testing.

2. The eye testing system of claim 1, wherein the telescopic arm includes a plurality of shafts adapted to telescopically move relative to each other to move the eye portion across a width of the acuity charts and a controller to control the horizontal telescopic movement of the arm and the bullseye.

3. The eye testing system of claim 1, wherein the bullseye is adapted to move in a direction perpendicular to a direction of telescopic movement of the telescopic arm.

4. The eye testing system of claim 1 further including a light source disposed to the top center of the acuity chart viewer for exposing an eye of a patient to light to facilitate in performing a dark adaptation test of the eye of the patient.

5. The eye testing system of claim 4, wherein the light source is a constant intensity light source.

6. The eye testing system of claim 4, wherein a level of intensity of light reaching to the eye of the patient is sequentially reduced by using a plurality of filters to perform the dark adaptation test of the eye.

7. The eye testing system of claim 4, wherein a level of intensity of light reaching to the eye of the patient is sequentially reduced by using a rheostat to perform the dark adaptation test of the eye.

8. The eye testing system of claim 7, wherein eye testing system includes a controller to control the rheostat to vary the level of intensity of light reaching the eye in discreet steps.

9. The eye testing system of claim 8, wherein a level of intensity of light reaching to the eye of the patient is automatically and sequentially reduced by varying an intensity of light emitted by the light source to perform the dark adaptation test of the eye.

10. The eye testing system of claim 1, wherein the plurality of acuity charts and the Snellen chart are digital charts suitable for use in computer applications.

11. The eye testing system of claim 1, wherein the bullseye is projected onto computer monitor screen utilizing a computer and a computer program for the mouse driver.

12. The eye testing system of claim 10 wherein the variable intensity light source is projected onto a computer monitor by a computer program.

13. An embodiment as in claim 1, wherein one or more magnifying glasses are placed between the eyepiece and the objective therein so to shrink the focal distances so that the system of eyepiece and objective assemblies may fit under one relatively small machine covering.

14. An embodiment wherein the filter eyepiece assembly therein is affixed to the head with sterilizable straps going around the head and a sterilizable, ergonomic interface between the forehead of the patient and the occlude base plate or the filter assembly is affixer to the front of headgear similar to an industrial eye protection googles.

15. A method claim to explain the procedure of testing for central loss of vision consisting of utilization of the special four part, all over pattern Diagram 100 eye chart and the delimiter arm to focus the vision on a small segment of the chart and causing the subject to establish the smallest letter that can be seen inside the bulls eye ring and then removing the bulls eye ring from the face of the chart and having the subject look all over the chart and try to see the lowest number that can be seen using side or peripheral vision and noting the acuity readings for the central vision versus the side or peripheral vision in the form of, for example, 20/60 central and 20/30 side and combining these two numbers into an index representing the denominators in the form 60/30 or an index of 2 wherein an index above 1 is suggestive of central vision loss.
